SEQUENCE LISTING (11 sequences)

SEQ ID NO: 1 - protein, 105 aa, Homo sapiens
ILPIASSCCTEVSHHISRRLLERVNMCRIQRADGDCDLAAVILHVKRRRICVSPHNHTVKQWMKVQAAKKNGKGNVCHRKKHHGKRNSNRAHQGKHETYGHKTPY

SEQ ID NO: 2 - protein, 77 aa, Homo sapiens
AVLPRSAKELRCQCIKTYSKPFHPKFIKELRVIESGPHCANTEIIVKLSDGRELCLDPKENWVQRVVEKFLKRAENS

SEQ ID NO: 3 - protein, 68 aa, Homo sapiens
KPVSLSYRCPCRFFESHVARANVKHLKILNTPNCALQIVARLKNNNRQVCIDPKLKWIQEYLEKALNK

SEQ ID NO: 4 - protein, 77 aa, Homo sapiens
SKCKCSRKGPKIRYSDVKKLEMKPKYPHCEEKMVIITTKSVSRYRGQEHCLHPKLQSTKRFIKWYNAWNEKRRVYEE

SEQ ID NO: 5 - protein, 97 aa, Homo sapiens
SLNPGVARGHRDRGQASRRWLQEGGQECECKDWFLRAPRRKFMTVSGLPKKQCPCDHFKGNVKKTRHQRHHRKPNKHSRACQQFLKQCQLRSFALPL

SEQ ID NO: 6 - DNA, 20 bp, Artificial Sequence
  antisense oligo
agtgggagagtgaggtggga

SEQ ID NO: 7 - DNA, 20 bp, Artificial Sequence
  antisense oligonucleotide
gccagcgttcccatttgagg

SEQ ID NO: 8 - DNA, 25 bp, Artificial Sequence
  siRNA top strand
guagcaaacagaagucaauaaauat

SEQ ID NO: 9 - RNA, 27 nt, Artificial Sequence
  siRNA bottom strand
auauuuauugacuucuguuugcuacuu

SEQ ID NO: 10 - DNA, 25 bp, Artificial Sequence
  siRNA top strand
gaaugugagugcaaagauugguucc

SEQ ID NO: 11 - RNA, 27 nt, Artificial Sequence
  siRNA bottome strand
ggaaccaaucuuugcacucacauucuu

(19) United States
(12) Patent Application Publication
(10) Pub. No.: US 2015/0140008 A1
(43) Pub. Date: May 21, 2015

(54) USES OF CXCL17, A NOVEL CHEMOKINE MARKER OF HUMAN LUNG AND GASTROINTESTINAL DISEASE

(71) Applicant: The Regents of the University of California, Oakland, CA (US)

(72) Inventors: Albert Zlotnik, San Diego, CA (US); Amanda M. Burkhardt, Long Beach, CA (US)

(73) Assignee: The Regents of the University of California, Oakland, CA (US)

(21) Appl. No.: 14/398,053

(22) PCT Filed: May 3, 2013
(86) PCT No.: PCT/US2013/039586

§ 371 Date: Oct. 30, 2014

Related U.S. Application Data
(60) Provisional application No. 61/642,209, filed on May 3, 2012.

(51) Int. Cl. C07K 16/24; C12N 15/113

(52) U.S. Cl. CPC C07K 16/24; C12N 15/1136; C12N 2310/11; C12N 2310/14; A61K 2039/505
USPC 424/145.1; 514/44 A

(57) ABSTRACT

A method of treating a disease associated with increased levels of chemokine CXCL17. The method includes administering to a subject in need of such treatment a therapeutically effective amount of a substance that lowers the level of CXCL17 activity. The substance can be a CXCL17 antibody, or an antisense compound targeting CXCL17 such as an antisense oligonucleotide or an siRNA. Also provided are methods of treating tumors with CXCL17 and methods of diagnosing a disease associated with increased levels of CXCL17.

### STATEMENT REGARDING FEDERALLY SPONSORED RESEARCH OR DEVELOPMENT

This invention was made with Government support under Grant No. 1R01A1093548-01A1 from the National Institute of Allergy and Infectious Diseases/National Institutes of Health. The Government has certain rights in this invention.

### REFERENCE TO SEQUENCE LISTING

This application contains an electronic form of a sequence listing. The contents of the sequence listing are incorporated herein by reference.

### BACKGROUND

1. Field of the Invention

The invention relates to methods and compositions for treating and diagnosing disorders involving the chemotactic protein CXCL17.

2. Related Art

The immune system is a complex network of cells that come together to defend the body from invading pathogens. These cells are dispersed throughout the body, yet must interact and communicate across these relatively large distances. To accomplish this, the cells communicate via soluble mediators called cytokines. Cytokines can induce the proliferation and differentiation of immune cells to establish effective immune responses. A subset of these secreted mediators are capable of inducing the chemotaxis of immune cells that express their cognate receptor. These chemotactic cytokines are called chemokines.

The human chemokine superfamily is comprised of 48 ligands and 19 known receptors [1,2]. Chemokine ligands have been divided into four subclasses based on the distribution of the four characteristic cysteine molecules within the protein: CC, CXC, C and CX3C [1,2]. Most of the chemokine ligands belong to the CC and CXC subclasses.

Chemokines are secreted in large amounts by cells so the soluble ligands can form a gradient to attract their target cells. If cells express the cognate receptor they will be able to sense the gradient and respond by migrating to the site of highest ligand concentration. All known chemokine receptors are G-Coupled Protein Receptors (GPCRs), which represent a large family of receptors whose primary function is to sense extracellular molecules and subsequently activate intracellular signal transduction pathways. Upon binding its appropriate ligand, the chemokine receptor GPCR activates the intracellular G protein, which begins the signaling cascade that eventually leads to chemotaxis of the cell [3-11].

Chemokines can be classified as either inflammatory or homeostatic based on their expression pattern. As their name suggests, inflammatory chemokines are produced when inflammatory stimuli are present, and therefore are important players in the development of immune responses [1,2]. A hallmark of these chemokines is the redundancy in the number of ligands that bind a single receptor [1,2]. It has been suggested that this redundancy allows for a stronger immune response; a wider range of cell types can be attracted by a larger number of ligands. The inflammatory chemokines have a lower level of conservation between species, which is likely a reflection of the fact that this class of chemokines is shaped by the infectious experience of a species [1]. For example, the inflammatory chemokine that recruits neutrophils, CXCL8, is present in humans but not in mice [2].

Conversely, homeostatic chemokines are conserved at a much higher rate between species. This class of chemokines is constitutively expressed and their expression either remains unchanged or decreases in the presence of inflammatory stimuli [1]. These ligands demonstrate higher receptor fidelity and attract cells critical in important cellular processes, including development and proper T:B cell interactions [12, 13]. The high conservation rate of homeostatic chemokines reflects their importance, from an evolutionary perspective [12].

### SUMMARY

In one aspect, a method of treating a disease associated with increased levels of chemokine CXCL17 is provided. The method includes administering to a subject in need of such treatment a therapeutically effective amount of a substance that lowers the level of CXCL17 activity. The substance that lowers the level of CXCL17 activity can be an anti-CXCL17 antibody, which can be a monoclonal antibody. In other embodiments, the substance can be an antisense compound targeting CXCL17, which can be an antisense oligonucleotide or an siRNA.

The disease can be an inflammatory disease or cancer. The inflammatory disease can be a disease of the lung, such as, but not limited to, chronic obstructive pulmonary disease (COPD), asthma, idiopathic pulmonary fibrosis, hypersensitivity pneumonitis, or non-specific interstitial pneumonia. Alternatively, the inflammatory disease can be a disease of the gut, such as but not limited to Celiac disease, Crohn's disease, ulcerative colitis, ulcers caused by Helicobacter pylori infection, irritable bowel syndrome, or rectal prolapse. The cancer can be small and non-small cell lung cancer, head and neck cancer, stomach cancer, colorectal cancer, pancreatic cancer, or hepatocellular carcinoma.

In another aspect, a method of treating a tumor in a subject in need of such treatment is provided. The method includes administering chemokine CXCL17 to the subject in an amount effective to increase macrophage numbers in the tumor. The tumor can be, but is not limited to, a colorectal, hepatocellular, pancreatic, glioblastoma, melanoma, soft tissue sarcoma, lymphoma, lung, breast carcinoma, prostate, bladder, head and neck, or ovarian tumor. Anti-tumor effects of administering the chemokine include, for example, tumor cell death, inhibition of tumor cell growth, inhibition of metastasis, decreased tumor size, and reversed or reduced malignant phenotype of tumor cells, or any combination thereof.

In a further aspect, a method of diagnosing a disease associated with increased levels of chemokine CXCL17 is provided. The method includes measuring the level of CXCL17 in a biological sample from a subject at risk for having the disease, and determining that the subject has the disease when the measured level is greater than a control level. The biological sample can be a biological fluid or a biological tissue. The control level can be an average or mean value of CXCL17 levels from a control population of one or more subjects without the disease. The disease can be an inflammatory disease or cancer. The inflammatory disease can be a disease of the lung, such as but not limited to chronic obstructive pulmonary disease (COPD), asthma, idiopathic pulmonary fibrosis, hypersensitivity pneumonitis, or non-specific interstitial pneumonia. Alternatively, the inflammatory disease can be a disease of the gut, such as but not limited to Celiac disease, Crohn's disease, ulcerative colitis, ulcers caused by Helicobacter pylori infection, irritable bowel syndrome, or rectal prolapse. The cancer can be small and non-small cell lung cancer, head and neck cancer, stomach cancer, colorectal cancer, pancreatic cancer, or hepatocellular carcinoma.

The subject in any of the methods can be a human or an animal. Animal subjects include, but is not limited to, mouse, rat, hamster, gerbil or guinea pig.

### BRIEF DESCRIPTION OF THE DRAWINGS

For a more complete understanding of the present invention, reference is now made to the following descriptions taken in conjunction with the accompanying drawings, in which:

FIG. 1 is a chart showing microarray data indicating that CXCL17 is a mucosal chemokine (A) Mean expression values (y axis) from microarray data for 105 normal human tissues from the BIGE (Body Index of Gene Expression) database. Data are displayed across the x-axis grouped in organ systems. Y axis is the mean expression values. Highlighted organ systems, which have the highest expression of CXCL17, are: DS, digestive system; RS, respiratory system; RT, reproductive tract. The values for the tissues with the highest expression of CXCL17 are shown in Table 1,

FIG. 2 is a drawing of Table 1 showing that the expression of CXCL17 is restricted to human mucosal tissues. Shown are the tissues with the highest average signal intensity values of the probeset corresponding to CXCL17 (226960_at) from the BIGE database that includes 105 different human tissues and cells. A graphic representation of these data is shown in FIG. 1. The highest expression of CXCL17 in the BIGE human gene expression database corresponds to mucosal tissues. The mean intensity signal is the averaged microarray signal from the replicates for each tissue included in the BIGE database.

FIG. 3 is a tissue section image showing that CXCL17 is expressed in the human lingual epithelium. Bar indicates 40 microns.

FIG. 4 is a tissue section image showing that CXCL17 is expressed in the human small intestine, in the epithelial cells that line the lumen of the intestine.

FIG. 5 is a tissue section image showing that CXCL17 is expressed in the cells that line the bronchoaveolar space. Bronchial wall 40X shows positive CXCL17 staining with heterogeneous intensity in the bronchial pseudostratified epithelium and in the mucous producing cells. There is also CXCL17 staining in endothelium and some macrophages and plasma cells.

FIG. 6 is a chart showing that CXCL17 is expressed in Hypersensitivity Pneumonitis and in Idiopathic Pulmonary fibrosis Bronchoalveolar lavage fluids. CXCL17 levels were detected by ELISA on bronchoalveolar lavage fluids of patients with these conditions.

FIG. 7A-7H is a panel of flow cytometry plots showing that intraperitoneal injection of recombinant CXCL17 increases recruitment of macrophages 48 hours post injection. After injecting rmCxcl17 into the peritoneal cavities of three mice, a small but significant increase in macrophages (Mφ), but not dendritic cells (DCs) was observed (F-H.) compared to mice injected with PBS vehicle (C-E.). The specific cell populations were determined by staining the cells with cell specific antibodies (F4/80, macrophages; CD11c, DCs; Gr-1, granulocytes). Unstained cells are also shown (A-B).

FIG. 8A-8D is a panel of flow cytometry plots showing that Cxcl17 (−/−) mice have a decreased percentage of F4/80⁺CD11b^{high} cells in the lungs compared to wild type (WT) mice. Cells collected from lungs of WT and Cxcl17 (−/−) mice were stained with fluorophore conjugated antibodies for analysis by flow cytometry. When the macrophage population (F4/80⁺CD11b^{high} cells) (B-D.) was analyzed, a significant decrease in the percentage of macrophages in Cxcl17 (−/−) lungs compared to WT mouse lungs was observed. Plots A-D are representative FACS plots from two separate experiments with n=5 per group.

FIG. 9 is a graph showing that Cxcl17(−/−) mice have decreased numbers of macrophages in the lungs compared to WT mice. The graph shows numbers of F4/80+CD11b+ cells recovered from lungs of WT or Cxcl17(−/−) mice. The latter mice have significantly less cells that express these markers, which characterize lung macrophages.

FIG. 10 is an Alignment of CXCL17 with other chemokines (mature peptides). Amino acid sequences of CCL28 (SEQ ID NO:1), CXCL8 (SEQ ID NO:2), CXCL12 (SEQ ID NO:3), CXCL14 (SEQ ID NO:4) AND CXCL17 (SEQ ID NO:5) are shown.

### DETAILED DESCRIPTION

The following is incorporated by reference herein: U.S. Provisional Patent Application No. 61/642,209, filed on May 3, 2012.

In one aspect, a method of treating a disease associated with increased levels of chemokine CXCL17 is provided. The method includes administering to a subject in need of such treatment a therapeutically effective amount of a substance that lowers the level of CXCL17 activity in the subject.

In the method, a therapeutically effective amount is an amount that promotes or enhances the well-being of the subject with respect to the medical treatment of his/her condition. For example, extension of the subject's life by any period of time, a decrease in pain to the subject that can be attributed to the subject's condition, a decrease in the severity of the disease, an increase in the therapeutic effect of a therapeutic agent, an improvement in the prognosis of the condition or disease, a decrease in the amount or frequency of administration of a therapeutic agent, an alteration in the treatment regimen of the subject that reduces invasiveness of treatment, and a decrease in the severity or frequency of side effects from a therapeutic agent. With respect to the treatment of cancer, therapeutic benefits also include a decrease or delay in the neoplastic development of the disease, decrease in hyperproliferation, reduction in tumor growth, delay of metastases, and reduction in cancer cell or tumor cell proliferation rate. The amount of active substance to be administered to the subject varies according to the weight of the subject, the mode of administration, and the indication and the severity of the disease, from which a skilled practitioner can determine a suitable dose.

In some embodiments, the substance that lowers CXCL17 activity levels in a subject is an anti-CXCL17 antibody. An anti-CXCL17 antibody is an antibody directed against CXCL17 that specifically binds to, or recognizes, the chemokine CXCL17. An anti-CXCL17 antibody can block or inhibit CXCL17 activity, thus lowering the overall level of CXCL17 activity in the subject's tissue or organ.

As used herein, an antibody can be any immunologic binding agent such as IgG, IgM, IgA, IgD and IgE. An antibody can also be any antibody-like molecule that has an antigen binding region, and includes antibody fragments such as Fab′, Fab, F(ab′)₂, single domain antibodies (DABs), Fv, scFv (single chain Fv), and the like. Techniques for preparing and using various antibody-based constructs and fragments are well known in the art. Means for preparing and characterizing antibodies are also well known in the art (See, for example, Harlow and Lane, “Antibodies: A Laboratory Manual,” Cold Spring Harbor Laboratory, 1988 [45]). Monoclonal antibodies (mAbs) are recognized to have certain advantages, e.g., reproducibility and large-scale production. Thus, monoclonal antibodies of the human, murine, monkey, rat, hamster, rabbit and even chicken origin, are contemplated.

Polyclonal antibodies against CXCL17 can be prepared in a wide range of animal species. Typically, the animal used for production of antisera is a rabbit, a mouse, a rat, a hamster, a guinea pig or a goat. To increase immunogenicity, use of adjuvants and conjugation to a carrier protein such as, but not limited to, keyhole limpet hemocyanin or bovine serum albumin are well known procedures.

A monoclonal antibody can be readily prepared through use of well-known techniques, such as those exemplified in U.S. Pat. No. 4,196,265, incorporated herein by reference [49-53]. Typically, this technique involves immunizing a suitable animal with a selected immunogen composition, e.g., a purified or partially purified polypeptide, peptide or domain. The immunizing composition is administered in a manner effective to stimulate antibody producing cells [54-56].

A polyclonal or monoclonal antibody can be further purified, if desired, using filtration, centrifugation and various chromatographic methods such as HPLC or affinity chromatography [56].

Humanized monoclonal antibodies are antibodies of animal origin that have been modified using genetic engineering techniques to replace constant region and/or variable region framework sequences with human sequences, while retaining the original antigen specificity. Such antibodies are commonly derived from rodent antibodies with specificity against human antigens. Such antibodies are generally useful for in vivo therapeutic applications. This strategy reduces the host response to the foreign antibody and allows selection of the human effector functions. Thus, humanized antibodies against CXCL17 are included in some embodiments of the invention, as are chimeric antibodies from mouse, rat, or other species, bearing human constant and/or variable region domains, bispecific antibodies, recombinant and engineered antibodies and fragments thereof. The techniques for producing humanized immunoglobulins are well known to those of skill in the art [53, 56-60]. For example U.S. Pat. No. 5,693,762 discloses methods for producing, and compositions of, humanized immunoglobulins having one or more complementarity determining regions (CDR's). When combined into an intact antibody, the humanized immunoglobulins are substantially non-immunogenic in humans and retain substantially the same affinity as the donor immunoglobulin to the antigen, such as a protein or other compound containing an epitope. Examples of other teachings in this area include U.S. Pat. Nos. 6,054,297; 5,861,155; and 6,020,192, all specifically incorporated by reference. Methods for the development of antibodies that are “custom-tailored” to the patient's disease are likewise known and such custom-tailored antibodies are also contemplated.

An anti-CXCL17 antibody can block or inhibit the chemotactic activity of CXCL17. The use of antibodies to inhibit the chemotactic activity of chemokines is widely demonstrated. For example, antibodies against chemokine CCL2 or CCL5 have been shown to inhibit chemotactic activity [61]. The ability of an anti-CXCL17 antibody to inhibit CXCL17 chemotactic activity can be determined by a transwell chemotaxis assay. This assay can be used to test the effectiveness of neutralizing antibodies against CXCL17. Briefly, the antibodies can be incubated with recombinant CXCL17 (rCXCL17) for 60 minutes prior to the start of the assay. The antibody/rCXCL17 solution can then be loaded into the bottom chamber of the transwell. Cells (primary or cell line) that have been previously shown to respond to rCXCL17 can be loaded into the top chamber of the transwell insert. Following incubation for several hours at 37° C., chemotaxis can be observed (via light microscopy) and the number of responding (chemotaxed) cells can be quantified via flow cytometry. The wells containing neutralizing antibodies will contain significantly fewer chemotaxed cells.

In some embodiments, the substance that lowers CXCL17 activity levels is an antisense compound targeting CXCL17. An antisense compound is an oligomeric or polymeric compound that can hybridize to a nucleic acid target via hydrogen bonding. One type of antisense compound is an antisense oligonucleotide. Another type of antisense compound is an siRNA.

As used herein, an antisense oligonucleotide is an oligomer or polymer of ribonucleic acid (RNA) or deoxyribonucleic acid (DNA) that can include naturally occurring nucleotides and/or modified or substituted oligonucleotides. In various embodiments, an antisense oligonucleotide includes a nucleotide sequence that hybridizes to the CXCL17 target sequence, and can include additional 5′ and/or 3′ flanking sequences, for example, for use as a primer binding site. In some embodiments, the antisense oligonucleotide can include modified oligonucleotide backbones such as, but not limited to, phosphorothioates, chiral phosphorothioates, phosphorodithioates, phosphotriesters, aminoalkylphosphotriesters, methyl and other alkyl phosphonates (e.g., 3′-alkylene phosphonates and chiral phosphonates), phosphinates, phosphoramidates (e.g., 3′-amino phosphoramidate and aminoalkylphosphoramidates), thionophosphoramidates, thionoalkylphosphonates, thionoalkyl phosphotriesters, and boranophosphates having normal 3′-5′ linkages, as well as 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included. References that teach the preparation of such modified backbone oligonucleotides are provided, for example, in U.S. Pat. Nos. 4,469,863 and 5,750,666, all incorporated by reference herein. The design and synthesis of antisense oligonucleotides is well known in the art [62]. Computer programs for the design of antisense oligonucleotide sequences are also available [63].

As used here, siRNA is a small inhibitory RNA duplex for use in RNA interference (RNAi) methods. RNAi is a naturally occurring gene-silencing process in which double-stranded RNA is cleaved to smaller double-stranded segments (siRNA), which then associate with a protein-RNA complex (called “RISC”) leading to cleavage of target mRNA [64]. In various embodiments, an siRNA can be 18-30 base pairs in size with varying degrees of complementarity to its target CXCL17 mRNA. In some embodiments, the siRNA can include unpaired bases at the 5′ and/or 3′ end of either or both the sense strand and antisense strand. The siRNA in some embodiments can be a duplex of two separate strands, or a single strand that forms a hairpin structure to form a duplex region. The design and synthesis of siRNAs is well known in the art [65]. Computer programs for the design of siRNAs are also available [66].

In another aspect, a method of treating a tumor in a subject in need of such treatment is provided. The method includes administering chemokine CXCL17 to the subject in an amount effective to increase macrophage numbers in the tumor. The inventors have found that CXCL17 is a chemotactic molecule for macrophages. Because macrophages play roles in immune and inflammatory responses, an increase in macrophages would lead to anti-tumor responses and other beneficial effects in the subject. In the method, CXCL17 can be obtained either commercially (for example, from](R&D Systems, Minneapolis, Minn., USA; or BioLegend, San Diego, Calif., USA) or can be prepared by expressing it in bacteria (E. coli) [39,40]. In some embodiments, the chemokine can be administered by direct injection into the tumor.

In a further aspect, a method of diagnosing a disease associated with increased levels of chemokine CXCL17 is provided. The method includes measuring the level of CXCL17 in a biological sample from a subject at risk for having the disease, and determining that the subject has the disease when the measured level is greater than a control level. The biological sample can be a biological fluid, a biological tissue, or a combination thereof. The biological fluid can be, but is not limited to, urine, feces, a bronchoalveolar lavage fluid, saliva, semen, vaginal fluid, or breast milk. The biological tissue can be, but is not limited to, lung, a tissue of the gastrointestinal tract, tongue, oral mucosa, vaginal or cervical tissue. Diagnosis of the disease is indicated when an increase in CXCL17 levels over control levels is at least two-fold and with a statistical significance of at least P<0.05.

The level of CXCL17 can be measure at the nucleic acid or protein level. For example, the amount of CXCL17 mRNA expressed in a cell can be measured, or the amount of CXCL17 protein present in a bronchoalveolar lavage fluid can be measured. Quantitation of mRNA can be performed using methods such as, but not limited to, PCR, microarray technologies, or Northern blots [46,47]. Quantitation of protein can be performed using immunodetection methods such as, but not limited to, enzyme linked immunosorbent assay (ELISA), radioimmunoassay (RIA), immunoradiometric assay, fluoroimmunoassay, chemiluminescent assay, bioluminescent assay, or Western blotting, FACS with anti-protein specific antibodies (for production by cells). In cases where RNA and protein levels are poorly correlated, CXCL17 protein measurement is envisioned. The control level can be an average or mean value of CXCL17 levels from a control population of one or more subjects without the disease. In some embodiments, a diagnosis that the subject has the disease can be followed by a treatment such as those described herein. For example, the diagnosis can be followed by a treatment that involves administering a steroid compound to a subject diagnosed with hypersensitivity pneumonitis, or by administering to a subject a therapeutically effective amount of a substance that lowers CXCL17 activity levels.

For the purposes of administration, the protein and nucleic acid compounds of the present invention may be formulated as pharmaceutical compositions. The compound can be present in the composition in an amount which is effective to treat a particular disorder of interest, and preferably with acceptable toxicity to the patient. A pharmaceutical composition of the present invention can also contain a pharmaceutically acceptable carrier. The carrier can be any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The amount of the compound administered will be dependent on the subject being treated, the subject's weight, the manner of administration and the judgment of the prescribing physician. Formulations can be prepared in an appropriate manner, and in accordance with accepted practices, by reference to procedures such as those disclosed in Remington's Pharmaceutical Sciences, Gennaro, Ed., Mack Publishing Co., Easton, Pa. 1990.

The route of administration can be oral, sublingual, buccal, nasal, inhalation, parenteral (including intraperitoneal, intraorgan, subcutaneous, intradermal, intramuscular, intra-articular, venous (central, hepatic or peripheral), lymphatic, cardiac, or arterial, or a combination thereof. Depending on the intended mode of administration, the pharmaceutical compositions may be in the form of solid, semi-solid or liquid dosage forms, such as, for example, tablets, suppositories, pills, capsules, powders, liquids, suspensions, ointments or lotions, preferably in unit dosage form suitable for single administration of a precise dosage. Intratumoral injection, or injection into the tumor vasculature is specifically contemplated for discrete, solid, accessible tumors. Local, regional or systemic administration also may be appropriate. For tumors of >4 cm, the volume to be administered can be about 4-10 ml, while for tumors of <4 cm, a volume of about 1-3 ml can be used.

For in vivo administration of nucleic acid compounds, the nucleic acid can be administered as a free (or “naked”) nucleic acid, or can be formulated with a delivery agent that increases delivery of the nucleic acid to a cellular target. Examples of delivery agents include, but are not limited to, liposomes, cationic lipids, PEGylated polycations, cationic block copolymers, and polyethyleneamine complexes [48].

CXCL17 was the final chemokine to be discovered [1,14]. Using protein threading techniques, CXCL17 was found to have a chemokine-like structure based on its structural similarity to CXCL8 and CXCL14 [14]. CXCL17 has 6 cysteines, including the 4 classic cysteines that anchor two disulfide bonds in the protein that are the hallmark of most other chemokines. In addition, CXCL17 has a glutamic acid between the first two cysteines, a factor that identified CXCL17 as a member of the CXC subfamily of chemokines. Several subsequent studies have shown a correlation of CXCL17 in cancer models, including hepatocellular carcinoma (HCC) and intraductal papillary mucinous neoplasm (IPMN) [15-17].

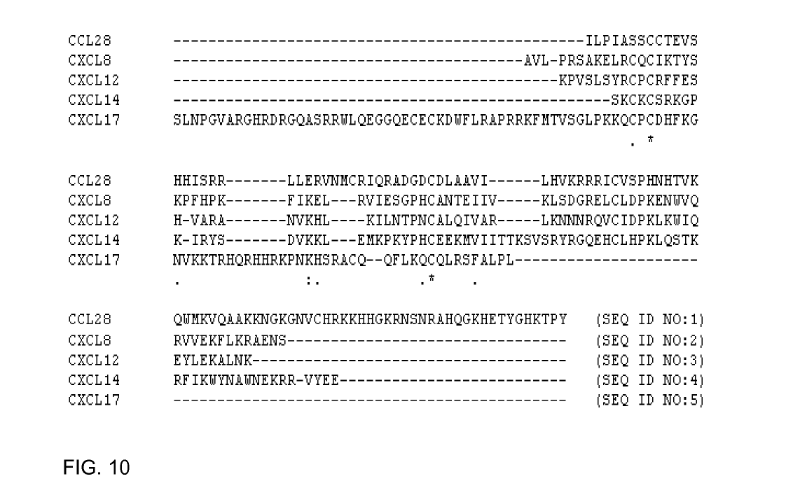

The amino acid sequence of CXCL17 shows 71% similarity to the mouse ortholog. It exhibits a CXCL8-like fold. The first 3 cysteines align with 3 cysteines in CXCL8, a reflection of the CXCL8-like fold of the protein. The CXCL17 structure has been compared to that of CCL5 and CCL4, both members of the CXCL-8 like fold family. Sequences of CXCL17 from various species have the following accession numbers (all incorporated by reference herein): HGNC:19232 (Human CXCL17) (HUGO Gene Nomenclature Committee database; Homologs: MGI:2387642 (mouse Cxcl17) (MGI database); RGD:1304717 (Rat Cxcl17) (RGD database); nucleotide sequence: RefSeq: NM198477 (NCBI Reference Sequence Database); protein sequence: UniProtKB:Q6UXB2 (UniProt Knowledgebase). A protein sequence alignment of CXCL17 compared to chemokines CCL28, CXCL8, CXCL12, and CXCL14 is shown in FIG. 10.

The inventors' studies represent the most in-depth characterization of CXCL17's functional chemotactic role in the immune system during both normal and diseased states. The inventors initially identified CXCL17 as an important mucosal chemokine by analyzing its expression in a comprehensive human gene expression database, called the Body Index of Gene Expression (BIGE). In this screen, we analyzed the expression of all 48 human chemokine ligands searching for those highly and specifically expressed in mucosal tissues. From this screen, three robustly and specifically expressed mucosal chemokines were identified: CCL28, CXCL14 and CXCL17. Both CCL28 and CXCL14 have been extensively studied and characterized for their chemotactic activities and their roles in human diseases. Furthermore, CCL28 had already been identified as a mucosal chemokine. Conversely, only three papers described CXC17 in the literature at the onset of the inventors' studies. Its expression pattern in the BIGE database indicated that CXCL17 was strongly and specifically expressed in the tissues of the gut and lung, with its highest sites of expression in the trachea, bronchus, and stomach. This pattern indicated that CXCL17 is an important mucosal chemokine of the digestive and respiratory systems.

The microarray expression data was confirmed using both quantitative real-time PCR (Q-PCR) and immunohistochemical (IHC) staining of normal human mucosal tissues. These experiments confirmed and expanded the observations from the BIGE database (microarray data).

Previous publications indicated that CXCL17 is structurally related to CCL28 and CXCL14 [1,14], two chemokines that have been shown to have antimicrobial activity [18-20]. Interestingly, the inventors found that CXCL17 has significant antimicrobial activity against a wide panel of microorganisms, indicating that this chemokine participates in shaping the microbiome of mucosal sites [21]. CXCL17 mediates this activity via direct disruption of the bacterial membranes [21]. These observations indicate that CXCL17 levels can influence the onset or progression of gut or lung diseases mediated by microorganisms such as ulcers triggered by infection with Helicobacter pylori [22].

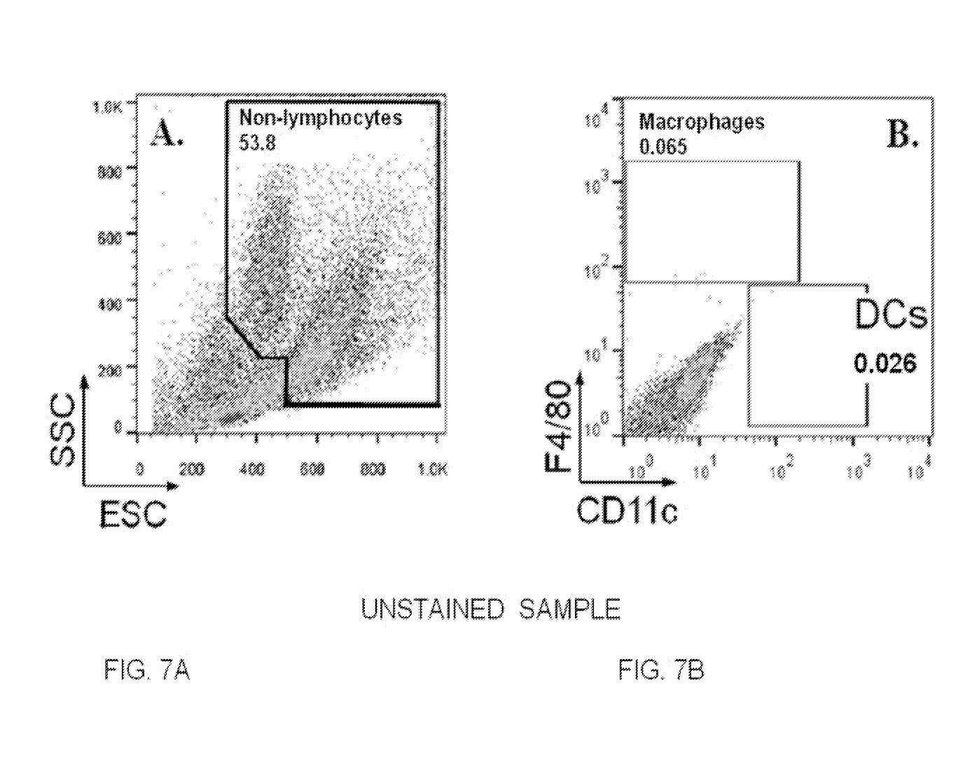

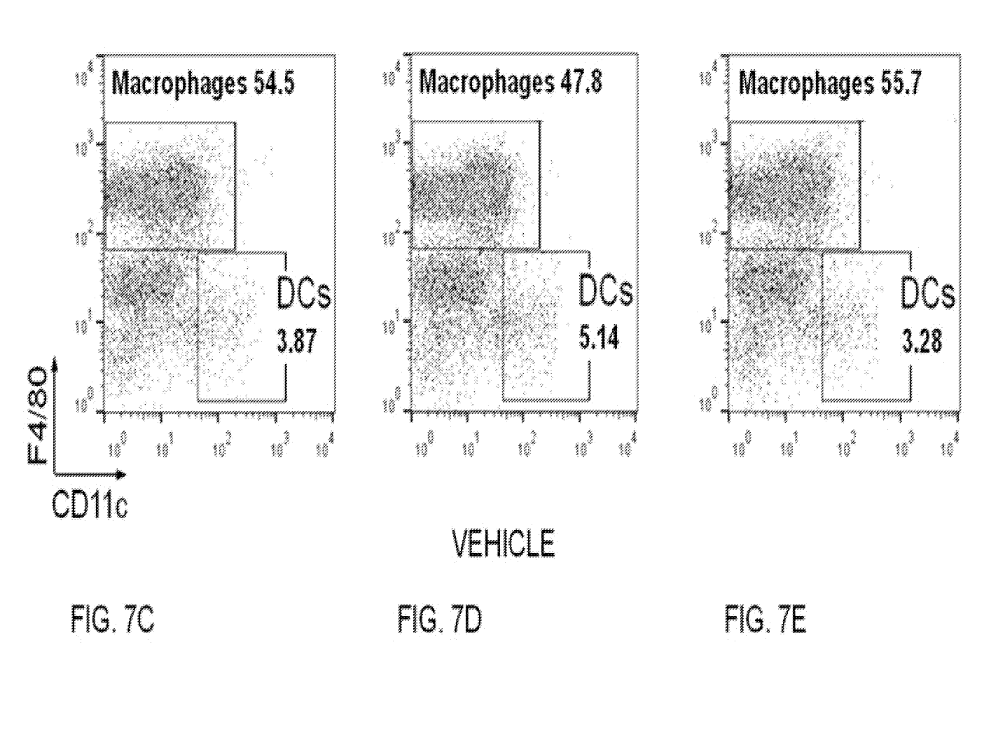

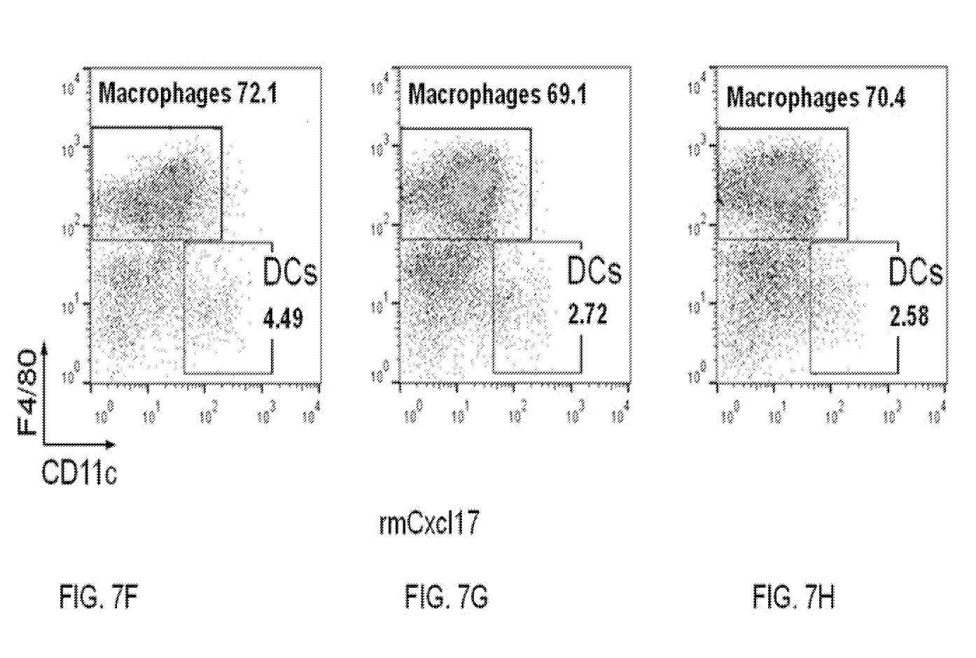

Chemotaxis assays were performed to determine which immune system cells specifically respond to CXCL17. These assays were performed both in vitro and in vivo. The in vitro assays utilized transwell chemotaxis assays to test both primary murine cells and cell lines for their chemotactic response to recombinant CXCL17. The results indicate that monocytic cell lines (monocytes, macrophages, dendritic cells) gave the chemotax in response to CXCL17. This chemotactic response is augmented by pretreatment of the cells with prostaglandin E-2 (PGE-2). Additionally, Pertussis toxin (PTX) treatment of these responding cell lines successfully abrogated chemotaxis, indicating that CXCL17 mediates chemotaxis through a class A GPCR that signals through Gαi, like other known chemokine receptors [7,8,23]. Further experiments of in vivo chemotaxis assays were performed by administering mice intraperitoneal injections of either CXCL17 or PBS (control saline solution) and then collecting and analyzing the cells recruited to the peritoneal cavity. The specific cell types recruited were determined by staining with cell lineage specific markers and analyzing the results by flow cytometry. From these experiments we found that the peritoneal cavities of mice which received the i.p. injection of CXCL17 contained a significantly higher number of macrophages after 48 hours than mice that were given the control i.p. injection. This indicates that CXCL17 mediates the recruitment of macrophages in vivo (FIG. 7).

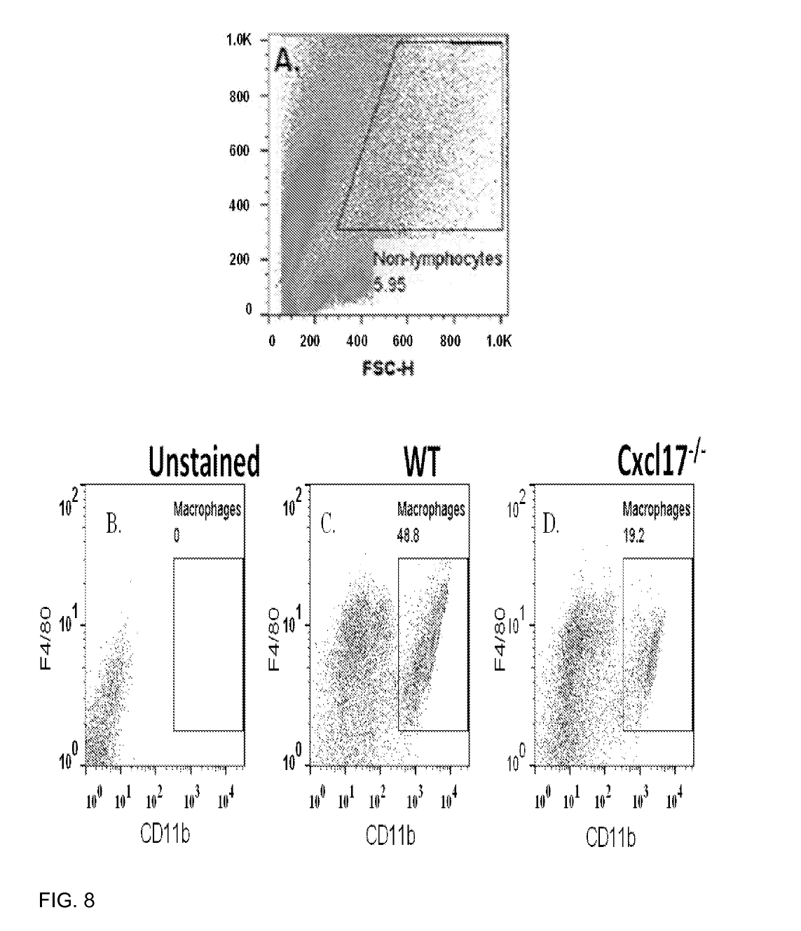

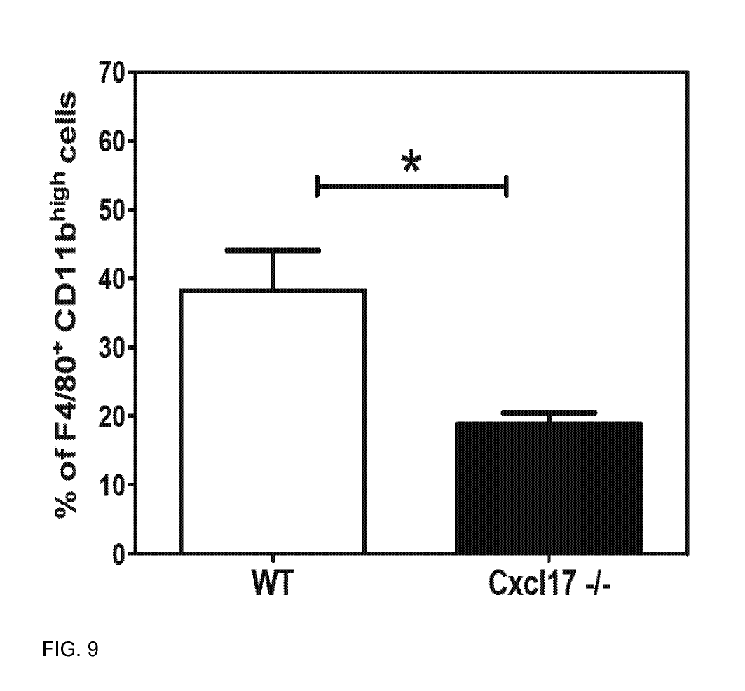

A Cxcl17 deficient (−/−) mouse was then analyzed for changes in cellular populations. This involved a comprehensive analysis of cell populations in immune organs or mucosal tissues of this mouse. It was found that the Cxcl17 (−/−) mice have significantly fewer lung macrophages compared to wild type (WT) mice (FIGS. 8 and 9). Taken together, these data indicate that CXCL17 is a major recruitment factor for some populations of macrophages that normally home to the lung tissues. These macrophages mediate various functions in vivo, from the initiation of immune responses to modulation of inflammation as well as immune surveillance against cancer or infectious agents.

CXCL17 was also measured using an ELISA assay the inventors developed using commercial reagents. It was observed that CXCL17 is significantly elevated in the bronchoalveolar lavage fluid (BALf) of patients with idiopathic pulmonary fibrosis (IPF) (compared to healthy subjects) [21]. Interestingly, a hallmark of this disease is a large increase in the number of macrophages recruited to the lung compared to other interstitial lung diseases [24,25].

Taken together, these data indicate that CXCL17 is one of the most important macrophage chemotactic factors and a major recruitment signal for macrophages to mucosal sites. Given the importance of macrophages as a central cell that controls and participates in inflammatory responses, these observations indicate that neutralizing the effects of CXCL17, for example, by administering to a patient an antibody against CXCL17, should alter the development or course of inflammatory responses in mucosal tissues including the lung and the gut.

Some chemokines have been identified as biomarkers of specific human diseases (11590198 [25,26], 22136974). Chemokines are specially suited to be excellent biomarkers because they are small, secreted proteins that can be easily measured by enzyme-linked immunosorbent assay (ELISA). Additionally, due to their mechanism of action, cells can produce chemokines at high concentrations in a short amount of time in order to produce a gradient. This, in conjunction with the high amount of specificity that chemokines exhibit in their association with various diseases makes this class of molecules excellent candidates to be biomarkers of human disease.

Given CXCL17's pattern of expression and the inventors' observations that CXCL17 is elevated in human lung diseases, CXCL17 can be used as a biomarker of human disease or a druggable target to treat disease.

As shown with microarray, gene expression, and immunohistochemical staining data, CXCL17 is robustly expressed in several mucosal tissues, specifically tissues of the gastrointestinal tract and the lungs (bronchus and trachea). Additionally, it has been shown that CXCL17 is an important macrophage chemotactic factor, suggesting that this chemokine is likely to be a key modulator of infiltration macrophages/monocytes in diseases where its expression levels are elevated. Based on these data, the modulation of CXCL17, either by modifying CXCL17 expression or by targeting its protein product, represents a novel therapeutic approach to treating diseases where CXCL17 is elevated.

The inventors have already found that CXCL17 is elevated above homeostatic levels in two human disease states: idiopathic pulmonary fibrosis (IPF) and ulcerative colitis (UC). Interestingly, a hallmark of IPF is a robust influx of macrophages to the lungs compared to other interstitial lung diseases [24]. Similarly, studies have suggested that macrophages may play an important role in the pathogenesis and development of UC [27-29]. There is still ample need for novel biologically based therapies for UC [30]. CXCL17 is a critical factor recruiting macrophages in these disease states. Therefore, targeting of the CXCL17 gene or protein product provides a new way to benefit patients suffering from these diseases. This is especially important given that there are no FDA approved therapies available for patients with IPF.

Therapies targeting CXCL17 are provided in two ways. In one embodiment, a monoclonal antibody specifically targeted against CXCL17 is used to prevent CXCL17 chemotactic activity, and therefore indirectly block the recruitment of macrophages. Monoclonal antibody therapy has been previously been shown to provide significant clinical benefit to patients; in fact, several therapeutic antibodies have been approved by the Food and Drug Administration (FDA) for indications such as rheumatorid arthritis, Crohn's disease and breast cancer [31-34]. Fully human antibodies can now be developed by those skilled in the art [35-38] as long as a particular antigen is known and available for the development of the monoclonal antibody. In the present case, CXCL17 can be obtained either commercially or can be prepared by expressing it in either bacteria (E. coli) [39,40]. Therapeutic monoclonal antibodies are typically administered intravenously (i.v.) and are disseminated throughout the body via the bloodstream. This route of delivery is relatively simple administration method that can be achieved rapidly. Additionally, an intratracheal instillation of this therapy can provide a site specific treatment for IPF. The antibody dosage can be about 1 mg/kg body weight to about 10 mg/kg body weight by intravenous infusion every 1-20 days [68].

In a second embodiment, CXCL17 is targeted at the gene expression level. Antisense therapy modulates the expression of a gene by blocking its effective transcription into mRNA by the cellular transcription machinery [41-43]. This therapeutic approach can prevent the successful production of any CXCL17 protein, therefore completely abolishing the capability of cells to be recruited to the disease sites. In some embodiments, this therapy can be administered i.v. or directly at the site of disease (lung/trachea, GI tract) to specifically block the CXCL17 mediated chemotactic response at these sites. The dosage can be about 1 to about 10 mg/kg body weight [69].

Recombinant CXCL17 can also be used as a therapy for cancers in a further embodiment. In this embodiment, tumors can be given direct injections of recombinant CXCL17 to increase the number of macrophages recruited to the tumor site [21]. These macrophages are expected to increase the antigen presenting cells taking up and presenting tumor antigens to immune cells, thereby augmenting the body's ability to fight the tumor [17]. Different macrophage subsets participate differentially in inflammatory responses [44]; thus, in another embodiment, the macrophages recruited by CXCL17 should be able to modify the inflammatory responses associated with tumors and enhance the body's resistance to tumor development. The CXCL17 dosage can be about 1-5 mg per intratumor administration [70].

The present invention may be better understood by referring to the accompanying examples, which are intended for illustration purposes only and should not in any sense be construed as limiting the scope of the invention.

### Example 1

### Assay

Bronchoalveolar lavage (BALf) samples from healthy or diseased human subjects were analyzed for CXCL17 by sandwich ELISA by coating 96-well plates (NUNC, Rochester, N.Y.) with primary monoclonal anti-human CXCL17 antibody (R&D Systems). Recombinant human CXCL17 (R&D Systems) was used as a standard. Bound standards and samples were detected by subsequent incubation with polyclonal anti-human CXCL17 antibody (R&D Systems) and horseradish peroxidase-conjugated mouse anti-human detection antibody (Abcam, Cambridge, Mass.). The binding was visualized using TMB (KPL, Gaithersburg Md.). The reaction was stopped with 2N H2SO4 and absorbance was read at 450 nm.

### Results

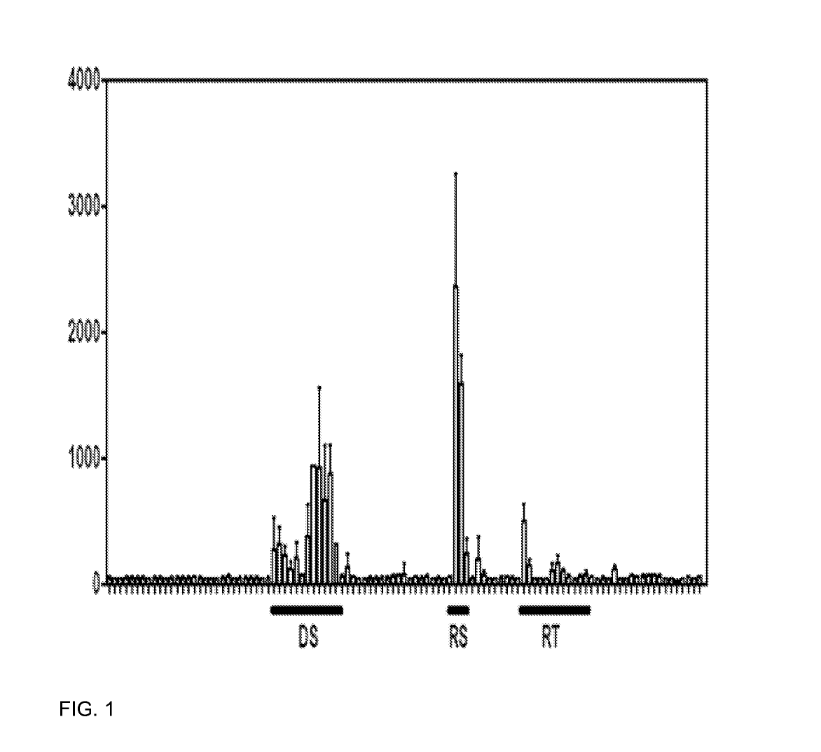

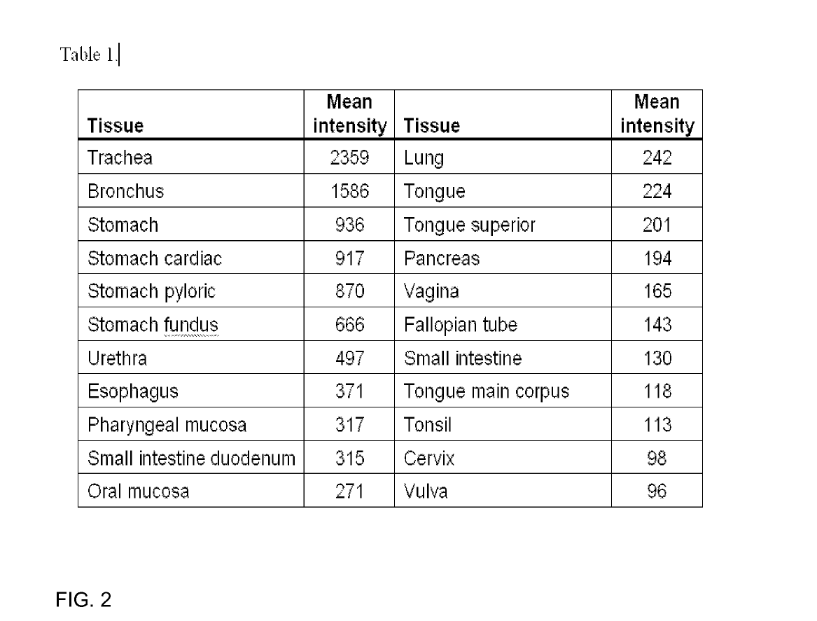

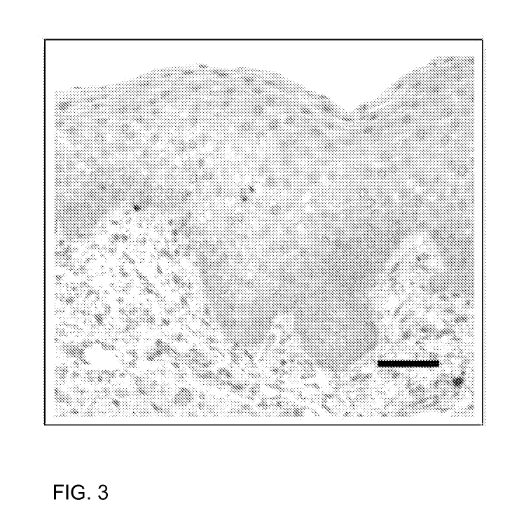

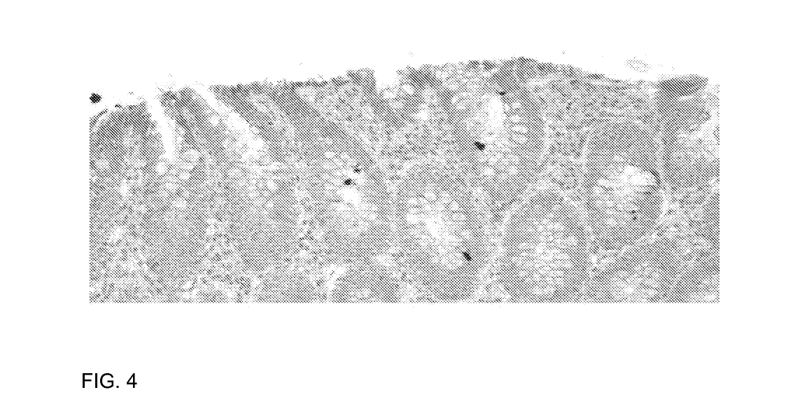

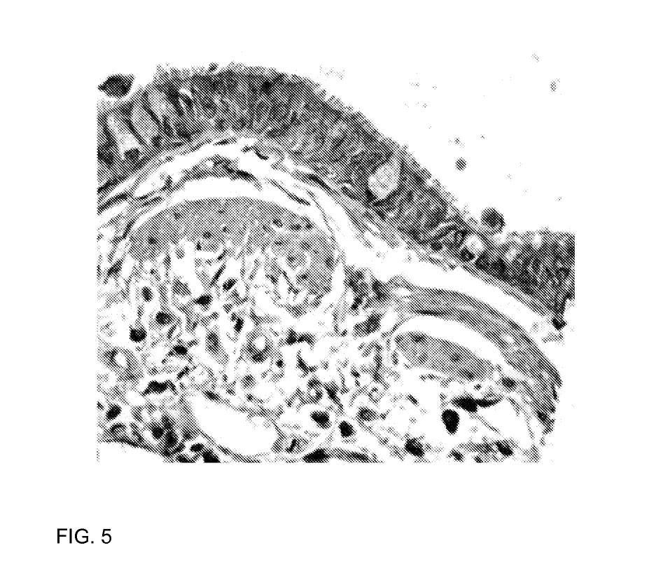

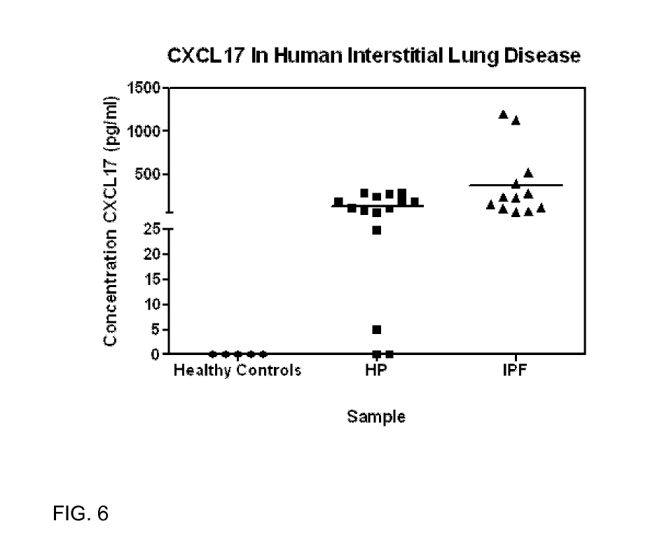

CXCL17 is the product of a gene that encodes a 13.8 kDa protein with the characteristic CXC chemokine fold [14]. The initial discovery of CXCL17 classified it as a chemokine that was expressed in the stomach and trachea [14]. The inventors have confirmed and expanded this observation. Microarray analysis, a novel approach to studying gene expression, was used to generate a comprehensive database of human gene expression in more than 105 normal human tissues and organs. Using a bioinformatics approach for the analysis, this database was screened for chemokines that were very robustly and specifically expressed within mucosal tissues. From this screen we identified CXCL17 as a mucosal chemokine, (FIGS. 1 and 2). In addition to being robustly expressed within specific mucosal tissues, CXCL17 has very discrete expression within those mucosal tissues. This is exemplified when CXCL17's expression is analyzed within the tongue: using a tongue microarray expression database it was found that CXCL17 is only expressed in the lingual epithelium (FIG. 3). When CXCL17's expression is analyzed in the microarray database, the inventers observed that its highest site of expression is the trachea and bronchus (FIGS. 1 and 5). The inventors have confirmed that CXCL17 is also expressed in the small intestine (FIG. 4). Based on this knowledge, the levels of CXCL17 in human bronchoalveolar lavage fluid (BALf) samples were analyzed from healthy controls and patients previously diagnosed with an interstitial lung disease (either hypersensitivity pneumonitis (HP) or interstitial pulmonary fibrosis (IPF)) (FIG. 6). Surprisingly, it was found that CXCL17 is elevated in patients with an interstitial lung disease compared to patients with no known lung disease. CXCL17 is more elevated in IPF compared to HP, suggesting that CXCL17 may play a different role in these two different interstitial lung disorders. Currently there are no reliable diagnostic methods to distinguish between HP and IPF. Given the high levels of CXCL17 in both of these disorders, and that CXCL17 is differentially detected between the two diseases, CXCL17 could serve as a new biomarker.

Patients with IPF typically present progressive dyspnea (difficulty breathing), and may also present cough and rales (a crackling sound in the lungs during inhalation). Differential diagnosis aims to exclude other potential causes that may present similar symptoms (asbestos exposure, chemotherapeutic drugs, rheumatoid arthritis, scleroderma, or hypersensitivity pneumonitis (HP), mixed connective tissue disease, or radiation induced fibrosis.

At present, there is no recognized satisfactory treatment, and therefore some of the efforts are directed instead to make sure that the patient does not have a different disease for which there are established treatments (i.e. HP). For HP, corticosteroids have been used for treatment. Another disease that should be excluded is non-specific interstitial pneumonia (NSIP).

Current studies testing potential treatments include cyclophosphamide, pirfenidone, and azathiopine. However, the severity of IPF is such that the prognosis is very poor. Most patients die within 3 years of diagnosis. Another more radical option includes lung transplant

The targeting of CXCL17 is contemplated to help in the treatment of these diseases. It has been observed that patients with these disorders have increased bacterial infections. Interestingly, the inventors have found that CXCL17 has antimicrobial activity. Therefore, alterations in CXCL17 levels in interstitial lung disease are contemplated to affect the susceptibility of the lungs of patients with these conditions to microbial infections, a finding that leads to the treatments provided herein.

### Example 2

### Intraperitoneal Injection of Recombinant CXCL17

After injecting 100 ng per mouse of recombinant mouse Cxcl17 into the peritoneal cavities of three mice, a small but significant increase in macrophages (Mφ), but not dendritic cells (DCs) was observed (Figure F.-H.) after 48 h compared to mice injected with PBS vehicle (Figure C.-E.). The specific cell populations were determined by staining the cells with cell specific antibodies (F4/80, macrophages; CD11c, DCs; Gr-1, granulocytes).

### Example 3

Referring to FIG. 8, lung cells were collected from Wild Type or Cxcl17 (−/−) mice. Lung lymphoid cells were obtained by disrupting the lung tissue mechanically and then treating it with collagenase. Single cells were collected after passing the resulting cell suspension through a sieve. Cells were then stained with fluorochrome-conjugated antibodies for analysis by flow cytometry. Lymphoid cells were identified by forward versus side scatter. When the macrophage population (F4/80+CD11bhigh cells) (B-D.) was analyzed, a significant decrease in the percentage of macrophages in Cxcl17 (−/−) lungs compared to WT mouse lungs was observed. Plots in FIG. 8 A-D are representative FACS plots from two separate experiments with n=5 per group.

FIG. 9 is a graph showing that Cxcl17(−/−) mice have decreased numbers of macrophages in the lungs compared to WT mice. The graph shows numbers of F4/80+CD11b+ cells recovered from lungs of WT or Cxcl17(−/−) mice. The latter mice have significantly less cells that express these markers, which characterize lung macrophages

### Example 4

For treatment of a patient, a monoclonal antibody against CXCL17 can be produced by immunizing a mouse with human CXCL17. Following several immunizations, the presence of anti-CXCL17 antibodies in the serum of the mouse can be assayed by testing the serum by enzyme-linked immunosorbant assay (ELISA). Once the presence of anti-CXCL17 antibodies is confirmed in the serum of a given mouse, its spleen can be fused to a myeloma cell suitable for the production of monoclonal antibodies using several techniques like PEG-driven fusion or electrical techniques. The resulting hybridomas can be selected in HAT medium and screened for the production of anti-CXCL17 antibodies by ELISA. Positive antibodies can also be tested for the neutralization activity by inhibition of CXCL17-driven chemotaxis of THP-1 cells. Anti-CXCL17 can be also screened by immunohistochemical staining of normal human trachea bronchus, tongue, colon, or small intestine. Antibodies can also be screened for their ability to inhibit CXCL17-driven calcium fluxes in THP-1 cells.

Mouse antibodies can be humanized by replacing their Fc region with human Fc. Alternatively, the binding site of the antibody can be sequenced and then molecular biology (e.g. cloning) techniques can be used to place the binding site in fully human antibodies. The expression of these antibodies can be performed in mammalian cell cultures and the antibodies can be purified by affinity chromatography. The purified antibodies can be tested for endotoxin content and otherwise can also be monitored for quality control using a Biacore instrument.

A given patient can be administered anti-CXCL17 monoclonal antibody in doses raging from 1-20 mg/kg body weight intravenously under medical supervision for 3 h to monitor adverse reactions. This dose/regimen can be repeated every 2 weeks-2 months. Expected results include reduced inflammatory responses, pain, swelling, altered blood counts for monocytes, reduction in inflammatory infiltrate, and reduction in disease score (symptoms).

### Example 5

A patient can be treated with an antisense compound targeting CXCL17. The methodology behind RNAi technology is to target and silence the expression of a specific gene [71]. This is accomplished when small, double stranded interfering RNAs (siRNAs) are processed into short, single strands. Using the cell's own RNA-induced silencing complex (RISC), the single strands will bind to the complementary target mRNA, which tags it for cleavage, thus preventing successful translation of that mRNA template [71]. In a therapeutic setting, RNAi therapy can be used to treat diseases, such as cancers or autoimmune diseases, by blocking specific gene expression programs that are required for tumor survival/growth or by blocking the recruitment or function of cells that mediate autoimmune diseases.

Tabernero et. al. recently used RNAi in a clinical trial to treat liver metastases of endometrial cancer [69] in a successful Phase I clinical trial. Similarly, Tekmira Pharmaceuticals Corporation is currently testing their RNAi therapy (a compound called TKM-PLK1 or TKM-080301, which targets PLK-1 (polo-like kinase 1) to treat solid tumors. Although the results of this Phase I clinical trial have not yet been published, the company is reporting favorable results (on the World Wide Web at tekmirapharm.com/Programs/Products.asp#plk1). RNAi therapy has also been reported to provide favorable outcomes in a Phase I clinical trial with melanoma patients [72].

Each of these studies used intravenous (i.v.) infusion as the route of administration for their therapeutic compound. The route of delivery for RNAi therapy is critical to ensure that the RNAi can inhibit successfully in an in vivo setting [71.72,69] utilized nanoparticles to deliver the RNAi to the cancers.

The primary challenge for effective gene silencing in vivo is delivery of the siRNA to the appropriate organ(s) with productive cellular uptake leading to engagement of RISC (RNA induced-silencing complex) in the cytosol. Successful delivery depends on siRNA formulations that confer ‘drug-like’ properties favorable to delivery and uptake following parenteral administration.

Lipid nanoparticles (LNP) can be used to deliver siRNAs and have been shown to silence gene expression in various species [73]. To use a CXCL17 siRNA, these can be designed as described [65] using the algorithms described in [66]. Similar approaches can be used with antisense oligonucleotides [62] using the algorithms described in [63]. The activity of these CXCL17 siRNAs can be tested in vitro by co-culture of the CXCL17 lipid nanoparticles with CXCL17-producing cells (like HEK-293 cells transfected with CXCL17 mRNA). The ability of the HEK-293 cells to produce CXCL17 can be measured by quantifying the levels of CXCL17 in the supernatant of these cell cultures.

Examples of antisense oligonucleotide sequences and siRNA sequences for CXCL17 are as follows:

a. Oligos synthesized to generate a phosphorothioate backbone denoted by *.

Site 1
(SEQ ID NO: 6)
5′ A*G*T*G*G*G*A*G*A*G*T*G*A*G*G*T*G*G*G*A 3′
Site 2
(SEQ ID NO: 7)
5′ G*C*C*A*G*C*G*T*T*C*C*C*A*T*T*T*G*A*G*G 3′

b. siRNA sequences for CXCL17; Upper case—RNA, lower case—DNA

Site 1
(SEQ ID NO: 8)
5′ GUAGCAAACAGAAGUCAAUAAAUat
(SEQ ID NO: 9)
UUCAUCGUUUGUCUUCAGUUAUUUAUA 5′
Site 2
(SEQ ID NO: 10)
5′ GAAUGUGAGUGCAAAGAUUGGUUcc
(SEQ ID NO: 11)
UUCUUACACUCACGUUUCUAACCAAGG 5′

siRNAs can be modified to reduce their immunostimulatory potential [74]. The lipid nanoparticles have a particle diameter of 80-100 nm which permits them to distribute around the peritoneum following parenteral administration [75]. These Cxcl17-Lipid nanoparticles (LNP) can be tested in a DSS model of colitis in mice as described [76], and the benefits of the therapy should be readily apparent by following the development (or not) of colitis in the treated mice.

For use in patients, the CXCL17-LNP can be used at a dose of (0.1-1.5 mg/kg body weight) through a central line as a 15 minute iv infusion via a controlled infusion device using an extension set with a 1.2 micron filter every 2 weeks, with a cycle of therapy defined as 2 doses given over one month. Patients can be premedicated with dexamethasone (20 mg iv), acetaminophen (650 mg orally), diphenhydramine 50 mg iv and 50 mg ranitidine iv to 30 minutes before infusion to reduce the risk of infusion-related reactions that can be observed with liposomal products.

The expected results include the amelioration of symptoms of the disease. In the case of lung interstitial or inflammatory diseases, patients would be expected to exhibit a gain of respiratory function. In the case of inflammatory digestive tract diseases, a reduction in the inflammation observed in various sites is expected. These parameters can be monitored by established techniques (for example, in the case of the gut: colonoscopy; in the case of the lung, x-rays and other radiology-based imaging techniques as well as physical measurements of lung function). The development of tumors can be monitored by CT scan.

### Example 6

For treatment of a tumor in a patient, CXCL17 can be produced by expression in a Bacullovirus expression system (insect cells). Following cloning of the CXCL17 gene into an appropriate expression vector, the cells are incubated at 37° C. CXCL17 protein production from the cells can be monitored by ELISA. CXCL17 activity can be evaluated by calcium flux and chemotaxis of THP-1 cells. Recombinant CXCL17 protein can be purified by high pressure liquid chromatography. The recombinant CXCL17 can be injected into tumor sites at 1 mg/kg body weight every 2 weeks-2 months. Tumor burden can be monitored by CT scans; macrophage infiltration can be monitored through biopsies. Macrophage content of the biopsies can be monitored by immunohistochemistry using macrophage-specific surface markers such as CD68. Expected results include shrinkage of tumor size, inhibition of metastatic foci, and overall improvement in the patient's quality of life.

In cancers of the digestive system (from the oral cavity to the colon) and lung, CXCL17 is expected to have a positive therapeutic effect by recruiting macrophages that will enhance anti-tumor effects including increased antigen presentation, tumor cytotoxicity and general immunosurveillance.

### REFERENCES

The following publications are incorporated by reference herein:
1. Zlotnik, A., O. Yoshie, and H. Nomiyama. 2006. The chemokine and chemokine receptor superfamilies and their molecular evolution. Genome Biol 7:243.
2. Zlotnik, A., and O. Yoshie. 2012. The chemokine superfamily revisited. Immunity 36:705-716.
3. Foord, S. M., T. I. Bonner, R. R. Neubig, E. M. Rosser, J. P. Pin, A. P. Davenport, M. Spedding, and A. J. Harmar. 2005. International Union of Pharmacology. XLVI. G protein-coupled receptor list. Pharmacol Rev 57:279-288.
4. Pierce, K. L., R. T. Premont, and R. J. Lefkowitz. 2002. Seven-transmembrane receptors. Nat Rev Mol Cell Biol 3:639-650.
5. Allen, S. J., S. E. Crown, and T. M. Handel. 2007. Chemokine: receptor structure, interactions, and antagonism. Annu Rev Immunol 25:787-820.
6. Wettschureck, N., and S. Offermanns. 2005. Mammalian G proteins and their cell type specific functions. Physiol Rev 85:1159-1204.
7. Thelen, M., P. Peveri, P. Kernen, V. von Tscharner, A. Walz, and M. Baggiolini. 1988. Mechanism of neutrophil activation by NAF, a novel monocyte-derived peptide agonist. FASEB J2:2702-2706.
8. Thelen, M. 2001. Dancing to the tune of chemokines. Nat Immunol 2:129-134.
9. Pitcher, J. A., N. J. Freedman, and R. J. Lefkowitz. 1998. G protein-coupled receptor kinases. Annu Rev Biochem 67:653-692.
10. Zlotnik, A., J. Morales, and J. A. Hedrick. 1999. Recent advances in chemokines and chemokine receptors. Crit Rev Immunol 19:1-47.
11. Horuk, R. 2001. Chemokine receptors. Cytokine Growth Factor Rev 12:313-335.
12. Knaut, H., C. Werz, R. Geisler, and C. Nusslein-Volhard. 2003. A zebrafish homologue of the chemokine receptor Cxcr4 is a germ-cell guidance receptor. Nature 421:279-282.
13. Zou, Y. R., A. H. Kottmann, M. Kuroda, I. Taniuchi, and D. R. Littman. 1998. Function of the chemokine receptor CXCR4 in haematopoiesis and in cerebellar development. Nature 393:595-599.
14. Pisabarro, M. T., B. Leung, M. Kwong, R. Corpuz, G. D. Frantz, N. Chiang, R. Vandlen, L. J. Diehl, N. Skelton, H. S. Kim, D. Eaton, and K. N. Schmidt. 2006. Cutting edge: novel human dendritic cell- and monocyte-attracting chemokine-like protein identified by fold recognition methods. J Immunol 176:2069-2073.
15. Weinstein, E. J., R. Head, D. W. Griggs, D. Sun, R. J. Evans, M. L. Swearingen, M. M. Westlin, and R. Mazzarella. 2006. VCC-1, a novel chemokine, promotes tumor growth. Biochem Biophys Res Commun 350:74-81.
16. Mu, X., Y. Chen, S. Wang, X. Huang, H. Pan, and M. Li. 2009. Overexpression of VCC-1 gene in human hepatocellular carcinoma cells promotes cell proliferation and invasion. Acta Biochim Biophys Sin (Shanghai) 41:631-637.
17. Hiraoka, N., R. Yamazaki-Itoh, Y. Ino, Y. Mizuguchi, T. Yamada, S. Hirohashi, and Y. Kanai. 2012. CXCL17 and ICAM2 are associated with a potential anti-tumor immune response in early intraepithelial stages of human pancreatic carcinogenesis. Gastroenterology 140:310-321.
18. Hieshima, K., H. Ohtani, M. Shibano, D. Izawa, T. Nakayama, Y. Kawasaki, F. Shiba, M. Shiota, F. Katou, T. Saito, and O. Yoshie. 2003. CCL28 has dual roles in mucosal immunity as a chemokine with broad-spectrum antimicrobial activity. J Immunol 170:1452-1461.
19. Liu, B., and E. Wilson. 2012. The antimicrobial activity of CCL28 is dependent on C-terminal positively-charged amino acids. Eur J Immunol 40:186-196.
20. Maerki, C., S. Meuter, M. Liebi, K. Muhlemann, M. J. Frederick, N. Yawalkar, B. Moser, and M. Wolf. 2009. Potent and broad-spectrum antimicrobial activity of CXCL14 suggests an immediate role in skin infections. J Immunol 182:507-514.
21. Burkhardt, A. M., K. P. Tai, J. P. Flores-Guiterrez, N. Vilches-Cisneros, K. Kamdar, O. Barbosa-Quintana, R. Valle-Rios, P. A. Hevezi, J. Zuniga, M. Selman, A. J. Ouellette, and A. Zlotnik. 2012. CXCL17 is a mucosal chemokine elevated in idiopathic pulmonary fibrosis that exhibits broad antimicrobial activity. J Immunol 188:6399-6406.
22. Malfertheiner, P., M. Selgrad, and J. Bornschein. 2012. Helicobacter pylori: clinical management. Curr Opin Gastroenterol 28:608-614.
23. Vassilatis, D. K., J. G. Hohmann, H. Zeng, F. Li, J. E. Ranchalis, M. T. Mortrud, A. Brown, S. S. Rodriguez, J. R. Weller, A. C. Wright, J. E. Bergmann, and G. A. Gaitanaris. 2003. The G protein-coupled receptor repertoires of human and mouse. Proc Natl Acad Sci USA 100:4903-4908.
24. Meyer, K. C., G. Raghu, R. P. Baughman, K. K. Brown, U. Costabel, R. M. du Bois, M. Drent, P. L. Haslam, D. S. Kim, S. Nagai, P. Rottoli, C. Saltini, M. Selman, C. Strange, and B. Wood. 2012. An official American Thoracic Society clinical practice guideline: the clinical utility of bronchoalveolar lavage cellular analysis in interstitial lung disease. Am J Respir Crit Care Med 185:1004-1014.
25. Pardo, A., K. M. Smith, J. Abrams, R. Coffman, M. Bustos, T. K. McClanahan, J. Grein, E. E. Murphy, A. Zlotnik, and M. Selman. 2001. CCL18/DC-CK-1/PARC up-regulation in hypersensitivity pneumonitis. J Leukoc Biol 70:610-616.
26. Berenguer, J., A. Fernandez-Rodriguez, M. A. Jimenez-Sousa, J. Cosin, P. Zarate, D. Micheloud, J. C. Lopez, P. Miralles, P. Catalan, and S. Resino. High plasma CXCL10 levels are associated with HCV-genotype 1, and higher insulin resistance, fibrosis, and HIV viral load in HIV/HCV coinfected patients. Cytokine 57:25-29.
27. Meuret, G., A. Bitzi, and B. Hammer. 1978. Macrophage turnover in Crohn's disease and ulcerative colitis. Gastroenterology 74:501-503.
28. Ahrens, R., A. Waddell, L. Seidu, C. Blanchard, R. Carey, E. Forbes, M. Lampinen, T. Wilson, E. Cohen, K. Stringer, E. Ballard, A. Munitz, H. Xu, N. Lee, J. J. Lee, M. E. Rothenberg, L. Denson, and S. P. Hogan. 2008. Intestinal macrophage/epithelial cell-derived CCL11/eotaxin-1 mediates eosinophil recruitment and function in pediatric ulcerative colitis. J Immunol 181:7390-7399.
29. Rahman, F. Z., A. M. Smith, B. Hayee, D. J. Marks, S. L. Bloom, and A. W. Segal. 2010. Delayed resolution of acute inflammation in ulcerative colitis is associated with elevated cytokine release downstream of TLR4. PLoS One 5:e9891.
30. Fiorino, G., M. Cesarini, and S. Danese. 2011. Biological therapy for ulcerative colitis: what is after anti-TNF. Curr Drug Targets 12:1433-1439.
31. Hudis, C. A. 2007. Trastuzumab—mechanism of action and use in clinical practice. N Engl J Med 357:39-51.
32. Moja, L., L. Tagliabue, S. Balduzzi, E. Parmelli, V. Pistotti, V. Guarneri, and R. D'Amico. 2012. Trastuzumab containing regimens for early breast cancer. Cochrane Database Syst Rev 4:CD006243.
33. Kempeni, J. 1999. Preliminary results of early clinical trials with the fully human anti-TNFalpha monoclonal antibody D2E7. Ann Rheum Dis 58 Suppl 1:170-72.
34. Rau, R. 2002. Adalimumab (a fully human anti-tumour necrosis factor alpha monoclonal antibody) in the treatment of active rheumatoid arthritis: the initial results of five trials. Ann Rheum Dis 61 Suppl 2:ii70-73.
35. Butler, M., and A. Meneses-Acosta. 2012. Recent advances in technology supporting biopharmaceutical production from mammalian cells. Appl Microbiol Biotechnol 96:885-894.
36. Rita Costa, A., M. Elisa Rodrigues, M. Henriques, J. Azeredo, and R. Oliveira. 2009. Guidelines to cell engineering for monoclonal antibody production. Eur J Pharm Biopharm 74:127-138.
37. Liu, H. F., J. Ma, C. Winter, and R. Bayer. 2010. Recovery and purification process development for monoclonal antibody production. MAbs 2:480-499.
38. Li, F., N. Vijayasankaran, A. Y. Shen, R. Kiss, and A. Amanullah. 2010. Cell culture processes for monoclonal antibody production. MAbs 2:466-479.
39. Qian, L., S. Zhu, J. Shen, X. Han, J. Gao, M. Wu, Y. Yu, H. Lu, and W. Han. 2012. Expression and purification of recombinant human Mig in Escherichia coli and its comparison with murine Mig. Protein Expr Purif 82:205-211.
40. Lu, H., M. Yu, Y. Sun, W. Mao, Q. Wang, M. Wu, and W. Han. 2007. Expression and purification of bioactive high-purity mouse monokine induced by IFN-gamma in Escherichia coli. Protein Expr Purif 55:132-138.
41. Forbes, D. C., and N. A. Peppas. 2012. Oral delivery of small RNA and DNA. J Control Release 162:438-445.
42. Spurgers, K. B., C. M. Sharkey, K. L. Warfield, and S. Bavari. 2008. Oligonucleotide antiviral therapeutics: antisense and RNA interference for highly pathogenic RNA viruses. Antiviral Res 78:26-36.
43. Dallas, A., and A. V. Vlassov. 2006. RNAi: a novel antisense technology and its therapeutic potential. Med Sci Monit 12:RA67-74.
44. Heine, G. H., A. Ortiz, Z. A. Massy, B. Lindholm, A. Wiecek, A. Martinez-Castelao, A. Covic, D. Goldsmith, G. Suleymanlar, G. M. London, G. Parati, R. Sicari, C. Zoccali, and D. Fliser. Monocyte subpopulations and cardiovascular risk in chronic kidney disease. Nat Rev Nephrol 8:362-369.
45. Harlow and Lane, “Antibodies: A Laboratory Manual,” Cold Spring Harbor Laboratory, 1988.
46. Pascal, L. E., et al., BMC Genomics 2008, 9:246.
47. Gry, M., et al., BMC Genomics 2009, 10:365.
48. Juliano, R. et al., Nucleic Acids Res. 2008 July; 36(12): 4158-4171.
49. Nakamura R M (1983) Monoclonal antibodies: methods and clinical laboratory applications. Clin Physiol Biochem 1: 160-172.
50. Leenaars M, Hendriksen C F (2005) Critical steps in the production of polyclonal and monoclonal antibodies: evaluation and recommendations. ILAR J 46: 269-279.
51. Tomita M, Tsumoto K (2011) Hybridoma technologies for antibody production. Immunotherapy 3: 371-380.
52. Siegel D L (2002) Recombinant monoclonal antibody technology. Transfus Clin Biol 9: 15-22.
53. Glassy M C (1993) Production methods for generating human monoclonal antibodies. Hum Antibodies Hybridomas 4: 154-165.
54. Butler M, Meneses-Acosta A (2012) Recent advances in technology supporting biopharmaceutical production from mammalian cells. Appl Microbiol Biotechnol 96: 885-894.
55. Rasmussen S K, Naested H, Muller C, Tolstrup A B, Frandsen T P (2012) Recombinant antibody mixtures: production strategies and cost considerations. Arch Biochem Biophys 526: 139-145.
56. Marichal-Gallardo P A, Alvarez M M (2012) State-of-the-art in downstream processing of monoclonal antibodies: process trends in design and validation. Biotechnol Prog 28: 899-916.
57. Chon J H, Zarbis-Papastoitsis G (2011) Advances in the production and downstream processing of antibodies. N Biotechnol 28: 458-463.
58. Di Fede G, Bronte G, Rizzo S, Rolfo Cervetto C, Cocorullo G, et al. (2011) Monoclonal antibodies and antibody fragments: state of the art and future perspectives in the treatment of non-haematological tumors. Expert Opin Biol Ther 11: 1433-1445.
59. Chiarella P (2011) Production, novel assay development and clinical applications of monoclonal antibodies. Recent Pat Anticancer Drug Discov 6: 258-267.
60. Kaneko E, Niwa R (2011) Optimizing therapeutic antibody function: progress with Fc domain engineering. BioDrugs 25: 1-11.
61. Castellani M L, Bhattacharya K, Tagen M, Kempuraj D, Perrella A, et al. (2007) Anti-chemokine therapy for inflammatory diseases. Int J Immunopathol Pharmacol 20: 447-453.
62. Sczakiel G (2000) Theoretical and experimental approaches to design effective antisense oligonucleotides. Front Biosci 5: D194-201.
63. Far R K, Leppert J, Frank K, Sczakiel G (2005) Technical improvements in the computational target search for antisense oligonucleotides. Oligonucleotides 15: 223-233
64. Bakhtiyari S, Haghani K, Basati G, Karimfar M H (2013) siRNA therapeutics in the treatment of diseases. Ther Deliv 4: 45-57.
65. Peek A S, Behlke M A (2007) Design of active small interfering RNAs. Curr Opin Mol Ther 9: 110-118.
66. Baranova A, Bode J, Manyam G, Emelianenko M (2011) An efficient algorithm for systematic analysis of nucleotide strings suitable for siRNA design. BMC Res Notes 4: 168.
67. Pisabarro M T, Leung B, Kwong M, Corpuz R, Frantz G D, et al. (2006) Cutting edge: novel human dendritic cell- and monocyte-attracting chemokine-like protein identified by fold recognition methods. J Immunol 176: 2069-2073.
68. Yan L, Anderson G M, DeWitte M, Nakada M T (2006) Therapeutic potential of cytokine and chemokine antagonists in cancer therapy. Eur J Cancer 42: 793-802.
69. Tabernero J, Shapiro G I, Lorusso P M, Cervantes A, Schwartz G K, et al. (2013) First-in-Humans Trial of an RNA Interference Therapeutic Targeting VEGF and KSP in Cancer Patients with Liver Involvement. Cancer Discov 3: 406-417.
70. Lechner M G, Russell S M, Bass R S, Epstein A L (2011) Chemokines, costimulatory molecules and fusion proteins for the immunotherapy of solid tumors. Immunotherapy 3: 1317-1340.
71. Gavrilov K, Saltzman W M (2012) Therapeutic siRNA: principles, challenges, and strategies. Yale J Biol Med 85: 187-200.
72. Davis M E, Zuckerman J E, Choi C H, Seligson D, Tolcher A, et al. (2010) Evidence of RNAi in humans from systemically administered siRNA via targeted nanoparticles. Nature 464: 1067-1070.
73. Wolfrum C, Shi S, Jayaprakash K N, Jayaraman M, Wang G, et al. (2007) Mechanisms and optimization of in vivo delivery of lipophilic siRNAs. Nat Biotechnol 25: 1149-1157.
74. Judge A D, Sood V, Shaw J R, Fang D, McClintock K, et al. (2005) Sequence-dependent stimulation of the mammalian innate immune response by synthetic siRNA. Nat Biotechnol 23: 457-462.
75. Senior J H (1987) Fate and behavior of liposomes in vivo: a review of controlling factors. Crit Rev Ther Drug Carrier Syst 3: 123-193.
76. Perse M, Cerar A (2012) Dextran sodium sulphate colitis mouse model: traps and tricks. J Biomed Biotechnol 2012: 718617.

Although the present invention has been described in connection with the preferred embodiments, it is to be understood that modifications and variations may be utilized without departing from the principles and scope of the invention, as those skilled in the art will readily understand. Accordingly, such modifications may be practiced within the scope of the invention and the following claims.

What is claimed is:

1. A method of treating a disease associated with increased levels of chemokine CXCL17, comprising administering to a subject in need of such treatment a therapeutically effective amount of a substance that lowers the level of CXCL17 activity.

2. The method of claim 1, wherein the substance is an anti-CXCL17 antibody.

3. The method of claim 2, wherein the antibody is a monoclonal antibody.

4. The method of claim 1, wherein the substance is an antisense compound targeting CXCL17.

5. The method of claim 4, wherein the antisense compound is an antisense oligonucleotide or an siRNA.

6. The method of claim 1, wherein the disease is an inflammatory disease or cancer.

7. The method of claim 6, wherein the inflammatory disease is a disease of the lung or gut.

8. The method of claim 7, wherein the disease of the lung or gut is chronic obstructive pulmonary disease (COPD), asthma, idiopathic pulmonary fibrosis, hypersensitivity pneumonitis, non-specific interstitial pneumonia, celiac disease, Crohn's disease, ulcerative colitis, ulcers caused by Helicobacter pylori infection, irritable bowel syndrome, or rectal prolapse.

9. The method of claim 6, wherein the cancer is small or non-small cell lung cancer, head and neck cancer, stomach cancer, colorectal cancer, pancreatic cancer, or hepatocellular carcinoma.

10. A method of treating a tumor in a subject in need of such treatment, comprising administering chemokine CXCL17 to the subject in an amount effective to increase macrophage numbers in the tumor.

11. The method of claim 10, wherein the tumor is a colorectal, hepatocellular, pancreatic, glioblastoma, melanoma, soft tissue sarcoma, lymphoma, lung, breast carcinoma, prostate, bladder, head and neck, or ovarian tumor.

12. A method of diagnosing a disease associated with increased levels of chemokine CXCL17, comprising
measuring the level of CXCL17 in a biological sample from a subject at risk for having the disease, and
determining that the subject has the disease when the measured level is greater than a control level.

13. The method of claim 12, wherein the biological sample is a biological fluid or a biological tissue.

14. The method of claim 12, wherein the disease is chronic obstructive pulmonary disease (COPD), asthma, idiopathic pulmonary fibrosis, hypersensitivity pneumonitis, non-specific interstitial pneumonia, celiac disease, Crohn's disease, ulcerative colitis, ulcers caused by Helicobacter pylori infection, irritable bowel syndrome, or rectal prolapse.

15. The method of claim 12, wherein the disease is small or non-small cell lung cancer, head and neck cancer, stomach cancer, colorectal cancer, pancreatic cancer, or hepatocellular carcinoma.

16. The method of claim 12, wherein the measuring comprises measuring CXCL17 at the nucleic acid or protein level.

17. The method of any one of the preceding claims, wherein the subject is a human or an animal.
